(19) United States
(12) Patent Application Publication
(10) Pub. No.: US 2022/0196573 A1
(43) Pub. Date: Jun. 23, 2022

(54) MEASUREMENT SYSTEM FOR ROCK VOLUME CHANGE UNDER MICROWAVE ACTION AND METHOD THEREOF

(71) Applicants: SHENZHEN UNIVERSITY, Shenzhen (CN); SICHUAN UNIVERSITY, Chengdu (CN)

(72) Inventors: Mingzhong GAO, Shenzhen (CN); Bengao YANG, Shenzhen (CN); Ruifeng TANG, Shenzhen (CN); Junjun LIU, Shenzhen (CN); Siqi YE, Shenzhen (CN); Xuemin ZHOU, Shenzhen (CN); Jun WANG, Shenzhen (CN); Haichun HAO, Shenzhen (CN); Zheng GAO, Shenzhen (CN); Yan WU, Shenzhen (CN); Zhaoying YANG, Shenzhen (CN); Xiangyue WEN, Shenzhen (CN); Xuan WANG, Shenzhen (CN)

(21) Appl. No.: 17/687,668

(22) Filed: Mar. 6, 2022

Related U.S. Application Data
(63) Continuation of application No. PCT/CN2021/105974, filed on Jul. 13, 2021.

(30) Foreign Application Priority Data
Dec. 17, 2020 (CN) 2020114967218

(51) Int. Cl. G01N 22/00; G01L 1/22; G01N 33/24

(52) U.S. Cl. CPC G01N 22/00; G01N 33/24; G01L 1/22

(57) ABSTRACT

A system and a method for measuring the volume change of rock under the action of microwaves are disclosed. The test cavity is of a sealed cavity structure, and a rock specimen is placed in the test cavity. The microwave control device is arranged inside the test cavity. The strain measuring device includes circumferential strain gauge and axial strain gauge and is attached to the surface of the rock specimen. The measurement circuit is connected with a number of strain measuring devices through lead wires, and the resistance strain gauge is connected with the measurement circuit. After the microwaves emitted by the microwave control device act on the rock specimen, data acquisition is carried out on the rock specimen through the number of circumferential strain gauges and the number of axial strain gauges which are attached to the surface of the rock specimen.

### CROSS-REFERENCE TO RELATED APPLICATIONS

This patent application is a continuation application of PCT/CN2021/105974, filed on Jul. 13, 2021, which claims the benefit and priority of Chinese Patent Application No. 202011496721.8, filed on Dec. 17, 2020, the disclosure of which is incorporated by reference herein in its entirety as part of the present application.

### FIELD OF THE INVENTION

The disclosure relates to the technical field of rock mechanics equipment, which relates to a rock volume change measurement system and a method thereof, and more specifically, to a measurement system and a method thereof capable of accurately measuring the influence of microwave action on the rock volume.

### BACKGROUND

For the mining of mineral resources within the deep surface, the effective breaking of the rocks has become a hot research topic. The traditional way is to use the drill bit to break the rock, which is limited by the factors such as excessive rock strength and easy wear of the drill bit, and has the problems of low efficiency and high cost.

Recently, microwaves have been introduced into the field of rock fragmentation. Microwave promotes the friction between the molecules in the rock to generate heat, which makes the overall temperature of the acted object rise, thus softening the rock mass for fragmentation, and has the advantages of no secondary pollution, which has been proved to be technically and economically feasible in this field. Relevant studies have proved that microwave can significantly reduce the strength of rock and even lead to rock melting and spalling, so microwave technologies have a good application prospect in the field of rock fragmentation.

Before applying microwave to the field of rock fragmentation, it is necessary to investigate the effect of microwave on the volume change of rock in the laboratory. Most of the existing methods for measuring the volume change of the rock indirectly measure the volume change of the rock through the volume changes of liquid or gas, and considering that the microwave action causes the temperature of the rock to rise suddenly and has influence on the volume changes of liquid or gas, rock volume measurement results in the existing arts are not reliable, and the industry urgently needs to propose a new rock volume change measurement scheme.

### SUMMARY

The technical problem to be solved by the present disclosure is to provide a system and a method for measuring the volume change of rock under the action of microwave, which can eliminate the volume change of liquid or gas caused by temperature change and more accurately measure the influence of microwave action on the volume of rock, in order to overcome the above defects in the prior art.

The technical scheme adopted by the disclosure for solving at least one of the technical problems is as follows.

A measurement system for rock volume change under the action of microwaves is disclosed. The system includes:

A test cavity of a sealed cavity structure, and a rock specimen is provided in the test cavity.

The microwave control device is provided inside the test cavity and is used for applying microwaves on the rock specimen.

A strain measure device, the strain measuring device includes a number of circumferential strain gauges and a number of axial strain gauges, and the number of strain measuring devices are attached to the surface of the rock specimen.

A measurement circuit and a resistance strain gauge; the measurement circuit is connected with a number of the strain measuring devices through lead wires, and the resistance strain gauge is connected with the measurement circuit.

Compared with the prior art, the technical scheme has the beneficial effects that after the microwaves emitted by the microwave control device act on the rock specimen, the data acquisition is carried out on the rock specimen through the number of circumferential strain gauges and axial strain gauges attached to the surface of the rock specimen, and the volume change of the rock specimen can be obtained by combining a measurement circuit and a resistance strain gauge, thereby eliminating the volume change of liquid or gas caused by temperature change and more accurately measuring the influence of the microwave action on the rock volume.

Furthermore, the strain measuring device includes a sensitive grid, a substrate and a covering layer.

The sensitive grid, the substrate and the covering layer form a sheet structure, the sensitive grid is arranged between the substrate and the covering layer and is connected with the measurement circuit through the lead wires.

When the strain measuring device is attached to the surface of a rock specimen, the substrate is provided close to the rock specimen, and the covering layer is provided far away from the rock specimen.

By adopting the scheme, the strain measuring device is formed by the sensitive grid, the substrate and the covering layer, and the sensitive grid is arranged between the substrate and the covering layer, so that the effect of protecting the sensitive grid can be achieved through the substrate and covering layer; and meanwhile, the strain measuring device is favorable for improving the measuring accuracy.

Furthermore, the covering layer is a foamed silicon dioxide layer.

The scheme has the beneficial effects that the foamed silicon dioxide layer is used as the covering layer, so that the effects of corrosion prevention and moisture prevention can be achieved, and the sensitive grid can be prevented from being subjected to microwave radiation.

Further, in the circumferential strain gauge, the sensitive grids are arranged along the horizontal direction; In the axial strain sheet, the sensitive grids are arranged along the vertical direction.

The adoption of the scheme has the beneficial effects that the circumferential deformation data and the axial deformation data of the rock specimen are collected, and the deformation data of the rock specimen is collected in multiple directions.

Further, the number of the circumferential strain gauges is 3 to 10, and the sensitive grids between the circumferential strain gauges are parallel to each other.

The number of the axial strain gauges is 3 to 10, and the sensitive grids among the axial strain gauges are parallel to each other.

The scheme has the beneficial effects that the number of the circumferential strain gauges and the number of the axial strain gauges can be adaptively adjusted according to the size of the rock specimen, so that the measurement accuracy can be improved.

Furthermore, the microwave control device includes a microwave source, a wave-guide component and a microwave emission disk, wherein one end of the microwave source is connected with the wave-guide component, and the other end of the microwave source is connected with the microwave emission disk.

The test cavity includes a bottom plate, a side plate and a top plate. A rock bearing abutment is arranged on the bottom plate of the test cavity, and a rock specimen is placed on the rock bearing abutment.

The microwave source is arranged on the top plate or the side plate of the test cavity, and the microwave emission disk emits microwaves towards the rock specimen.

The adoption of the scheme has the advantages that the rock bearing abutment is arranged on the bottom plate of the test cavity, the microwave source is arranged on the top plate or the side plate of the test cavity, and the relative position relation between the microwave source and the rock specimen can be adjusted according to actual conditions, so that the universality of the system is improved.

Further, the microwave source includes a power supply assembly, a transformer assembly, and a control circuit.

By adopting the scheme, the invention has the beneficial effects that the microwave source is formed by the power supply assembly, the transformer assembly and the control circuit, so as to provide the microwave for the test.

Further, the bottom plate, the side plate and the top plate are of a plate-shaped double-layer structure consisting of a metal layer cavity and a heat insulation layer cavity.

The side plate is provided with a pick-and-place through hole, and the pick-and-place through hole is provided with the side door.

The rock bearing abutment is arranged on the bottom plate through a height adjust assembly.

By adopting the scheme, the invention has the advantages that the bottom plate, the side plates and the top plate are formed by the metal layer cavity and the heat insulation layer cavity, namely, the whole test cavity is of a double-layer structure, so that the effects of heat insulation and radiation prevention can be better achieved, and the safety of a test process is ensured.

Further, the measurement circuit includes an amplifier and a bridge.

One end of the lead is connected with the strain measuring device, and the other end of the lead is connected with the bridge through the amplifier; and the bridge is connected with a resistance strain gauge.

The technical scheme has the beneficial effects that the electric signals acquired by the strain measuring device are processed by the amplifier and the bridge, which is beneficial to improving the reliability of test data.

The invention relates to a method for measuring a rock volume change under the action of microwaves, which is based on the rock volume change measurement system and includes the following steps:

Emitting, by the microwave control device, microwaves to the rock specimen.

Acquiring a first resistance variation through the circumferential strain gauge and the measurement circuit, acquiring, the second resistance variation through the axial strain gauge and the measurement circuit and inputting the acquired first resistance variation and second resistance variation into the resistance strain gauge after the microwaves act on the rock specimen.

Obtaining, by the resistance strain gauge, the rock circumferential volume strain according to the first resistance variation, the rock axial volume strain according to the second resistance variation, and the total volume application amount according to the rock circumferential volume strain and the rock axial volume strain.

Compared with the prior art, the technical scheme has the beneficial effects that after the microwaves emitted by the microwave control device act on the rock specimen, the data acquisition is carried out on the rock specimen through the number of circumferential strain gauges and axial strain gauges attached to the surface of the rock specimen, and the volume change of the rock specimen can be obtained by combining a measurement circuit and a resistance strain gauge, thereby eliminating the volume change of liquid or gas caused by temperature change and more accurately measuring the influence of the microwave action on the rock volume.

### BRIEF DESCRIPTION OF THE DRAWINGS

FIG. 1 is an overall schematic diagram of a measurement system for rock volume change under the action of microwaves according to the present disclosure.

FIG. 2 is a schematic diagram of a microwave control device in the measurement system for rock volume change under the action of microwaves according to the disclosure.

FIG. 3 is a schematic diagram of a strain measuring device in the system for measuring the volume change of rock under the action of microwave according to the disclosure.

FIG. 4 is a schematic diagram of a measurement circuit in the system for measuring the volume change of rock under the action of microwave according to the disclosure.

FIG. 5 is a flow chart of a method for measuring rock volume change under the action of microwaves according to the disclosure.

In the drawings, the components represented by the numbers are listed as follows:

test cavity 1, microwave control device 2, strain measurement device 3, measurement circuit 4, lead wire 5, resistance strain gauge 6, rock bearing abutment 101, height adjusting assembly 102, microwave source 201, wave-guide assembly 202, microwave emission disk 203, circumferential strain gauge 301, axial strain gauge 302, sensitive grid 303, substrate 304, amplifier 401, and bridge 402.

### DETAILED DESCRIPTION OF THE EMBODIMENTS

In order to make the purpose, technical solution and advantages of the present invention more clear and definite, the following is a further detailed description of the present invention with reference to the accompanying drawings and examples. It should be understood that the specific embodiments described herein are merely illustrative of the invention and are not intended to limit the disclosure.

In the description of the disclosure, it should be understood that the orientation or positional relationship indicated by the terms “center”, “upper”, “lower”, “front”, and “rear” as well as “left” and “right” are based on the orientation or positional relationship shown in the drawings, and are only for convenience of description of the disclosure and simplification of description, it is not intended to indicate or imply that the devices or components referred to must have a particular orientation, be constructed and operate in a particular orientation, and therefore should not be construed as limiting the invention. Furthermore, the terms “first” and “second” are used for descriptive purposes only and are not to be construed as indicating or implying relative importance.

In the description of the present invention, it should be noted that, unless otherwise specified and limited, the terms “mounting”, “connecting” and “connecting” should be interpreted in a broad sense, for example, they may be fixedly connected, detachably connected or integrally connected, mechanically connected or electrically connected; directly connected or indirectly connected through an intermediate medium. It can be the internal communication of two components. When an element is referred to as being “fixed to” or “disposed on” another element, it may be directly on the other element or an intervening element may also be presented. When an assembly is considered to be “connected” to another element, it may be directly connected to the other element or there may be an intervening element. Those of ordinary skill in the art will understand the specific meaning of the above terms in the context of the disclosure.

Before applying microwave to the field of rock fragmentation on a large scale, it is necessary to explore the effect of microwave on rock volume change in the laboratory. In the existing arts, the volume change of the rock is indirectly measured through the volume change of liquid or gas, and considering that the microwave action causes the temperature of the rock to rise suddenly and has an influence on the volume changes of liquid or gas, As a result, existing rock volume measurement are thus not reliable. Briefly, in the existing arts, when the volume change of the rock is study, the volume change of the surrounding liquid or gas cause by the rock volume change is used to indirectly measure the rock volume change. For example, a rock specimen is placed in a container filled with liquid, and when the volume of the rock increases, parts of the surrounding liquid is discharged, and the existing are is to indirectly measure the volume change of the rock by measuring the volume of the discharged liquid. However, when the microwave is applied on the rock specimen, the microwave will affect the volume of the liquid, and it is difficult for the volume of the discharged liquid to accurately reflect the volume change of the rock, which is the limitation of the prior art. Therefore, there is an urgent need to propose a new measurement scheme for rock volume change.

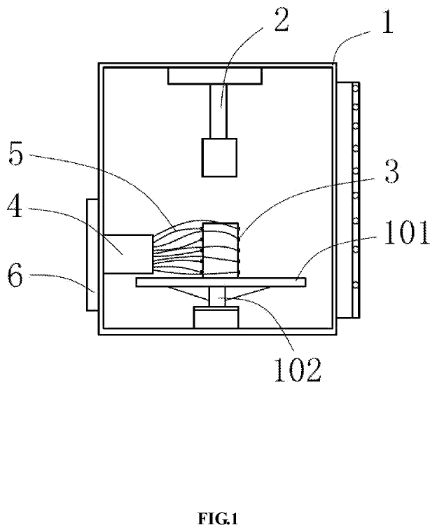

As shown in FIG. 1, a measurement system for rock volume change under the action of microwave includes a test cavity 1, a microwave control device 2, a strain measurement device 3, a measurement circuit 4 and a resistance strain gauge 6.

The test cavity 1 is of a sealed cavity structure, and a rock specimen is provided in the test cavity 1. The microwave control device 2 is arranged inside the test cavity 1, and the microwave control device 2 is used for applying microwaves on the rock specimen. The strain measuring device 3 includes a circumferential strain gauge 301 and an axial strain gauge 302. A number of circumferential strain gauges 301 and a number of axial strain gauges 302 are respectively provided. The number of strain measuring devices 3 are attached to the surface of the rock specimen. The measurement circuit 4 is connected to the number of the strain measuring devices 3 through lead wires 5, and the resistance strain gauge 6 is connected to the measurement circuit 4.

The core of the present disclosure is that a number of circumferential strain gauges 301 and a number of axial strain gauges 302 are used as the strain measurement device 3 to directly measure the volume change of the rock specimen. The number of circumferential strain gauges 301 are arranged on the rock specimen, and annular deformation data on the circumferential strain gauges 301 are collected. Meanwhile, a number of axial strain gauges 302 are arranged on the rock specimen for obtaining the axial deformation data thereon. The integral deformation condition of the rock specimen can be accurately reflected through the circumferential deformation data and the axial deformation data.

Therefore, based on the above technical solution, after the microwaves transmitted by the microwave control device 2 application on the rock specimen, the number of circumferential strain gauges 301 and the number of axial strain gauges 302 attached to the surface of the rock specimen are used to acquire data of the rock specimen, and the measurement circuit 4 and the resistance strain gauge 6 are used to obtain the volume change of the rock specimens, thereby eliminating the volume change of liquid or gas caused by temperature change and more accurately measuring the influence of the microwave action on the rock volume.

Preferably, the strain measurement device 3 includes a sensitive grid 303, a substrate 304, and a covering layer. The sensitive grid 303, the substrate 304, and the covering layer form a sheet-like structure. The sensitive grid 303 is arranged between the substrate 304 and the covering layer, and is connected to the measurement circuit 4 through a lead wire 5. When the strain measurement device 3 is attached to the surface of the rock specimen, the substrate 304 is arranged close to the rock specimen and the overlay is located away from the rock specimen. The strain measurement device 3 includes the sensitive grid 303, the substrate 304 and the covering layer. The sensitive grid 303 is arranged between the substrate 304 and the covering layer, so that the substrate 304 can protect the sensitive grid 303 and improve the measurement accuracy.

During the test, the sensing grid 303 may be affected with moisture or even corroded, resulting in failure of the strain measurement device 3. In order to avoid the problem, it is preferred that the cover layer is a foamed silicon dioxide layer. The foam silicon dioxide layer is used as a covering layer, which can play a role in preventing corrosion and moisture, and can also prevent that sensitive grid 303 from being subject to microwave radiation. As such, a foamed silica sleeve is provided on the outside of the lead wire 5, and the lead wire 5 is protected from moisture and corrosion by the foamed silica sleeve.

Specifically, the substrate 304 is an insulating substrate 304. The sensitive grid 303 is pasted on the substrate 304. The sensitive grid 303 is used to convert the strain of the rock specimen into the resistance variation. In this process, the substrate 304 is arranged between the sensitive grid 303 and the rock specimen, which can play an insulating role. In addition, the covering layer is made of an organic polymer material, the sensitive grid 303 is protected from radiation damage and mechanical damage, and has good mechanical properties. The lead wire 5 is connected to the sensing grid 303 and the measurement circuit 4, and a double-lead wire 5 and a multi-point welding method are adopted.

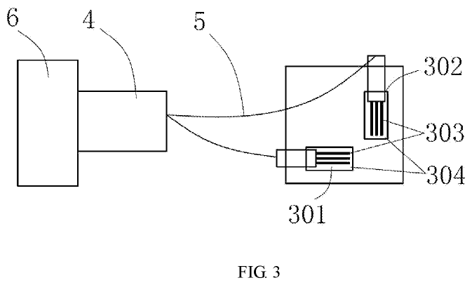

As shown in FIG. 3, preferably, in the circumferential strain gauge 301, the sensitive grids 303 are arranged along the horizontal direction. In the axial strain gauge 302, the sensitive grids 303 are arranged along the vertical direction. The annular deformation data of the rock specimen are acquired through the horizontally arranged sensitive grids 303, and the axial deformation data are acquired through the vertically arranged sensitive grids 303. The deformation data of the rock specimen are collected in multiple directions. Specifically, the number of the circumferential strain gauges 301 is 3-10, and the sensitive grids 303 between the circumferential strain gauges 301 are parallel to each other. The number of the axial strain gauges 302 is 3-10, and the sensitive grids 303 between the axial strain gauges 302 are parallel to each other. Based on the size of the rock specimen, the number of the circumferential strain gauges 301 and the number of the axial strain gauges 302 are adaptively adjusted, which is beneficial to improving the measurement accuracy.

For example, when the size of the rock specimen is 50 mm in diameter and 100 mm in height, five circumferential strain gauges 301 are provided and five axial strain gauges 302 are provided. Considering that the mineral composition and internal structure of the rock specimen are complex and diverse, and the thermal volume expansion of each part may be inconsistent, in order to maintain the accuracy of the measured volume change as much as possible and take into account the actual needs, a rock specimen with a diameter of 50 mm and a height of 100 mm is divided into five segments, and the strain of each segment is measured respectively during the test. Strain gauges are arranged at the midpoint of each segment to measure the circumferential and axial strains of the segment. The deformation of each segment is approximated as uniform deformation, so as to measure the overall volume changes.

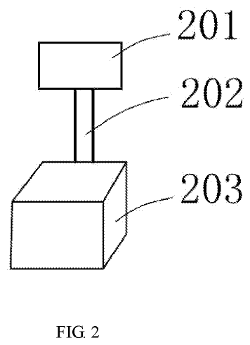

As shown in FIG. 1 and FIG. 2, the microwave control device 2 includes a microwave source 201, a wave-guide assembly 202, and a microwave emission disk 203. One end of the microwave source 201 is connected to the wave-guide assembly 202, and the other end of the microwave source 201 is connected to the microwave emission disk 203. The test cavity 1 includes a bottom plate, a side plate and a top plate. A rock bearing abutment 101 is arranged on the bottom plate of the test cavity 1. A rock specimen is placed on the rock bearing abutment 101. The microwave source 201 is arranged on a top plate or a side plate of the test cavity 1, and the microwave emission disk 203 emits microwaves toward the rock specimen. The bottom plate of the test cavity 1 is provided with a rock bearing abutment 101, the microwave source 201 is arranged on the top plate or the side plate of the test cavity 1. The relative position relationship between the microwave source 201 and the rock specimen can be adjusted according to the actual situation, so as to improve the universality of the system. Specifically, the microwave source 201 includes a power supply assembly, a transformer assembly and a control circuit, and the microwave source 201 includes the power supply assembly, the transformer assembly and the control circuit, so as to provide microwaves for testing.

The microwave control device 2 includes a microwave source 201, a wave-guide assembly 202, and a microwave emission disk 203, which converts electric energy into microwave energy, and heats rocks by transmitting microwaves to achieve the effect of rapid rock breaking. The circumferential strain gauges 301 and the axial strain gauges 302 are stuck on the rock specimen, and each strain gauge is uniformly distributed at the midpoint of each section of the rock specimen. The strain gauge can simultaneously measure the circumferential strain and the axial strain.

The bottom plate, the side plate and the top plate are in a plate-shaped double-layer structure consisting of a metal layer cavity and a heat insulation layer cavity. The side plate is provided with a pick-and-place through hole, and the pick-and-place through hole is provided with the side door. The rock bearing abutment 101 is arranged on the floor by a height adjusting assembly 102. The bottom plate, the side plate and the top plate are composed of the metal layer cavity and the heat insulation layer cavity, namely, the whole test cavity 1 is of a double-layer structure, which can play a better role in heat insulation and radiation prevention and ensure the safety of the test process. A height adjusting assembly 102 is arranged to conveniently adjust the height of the rock bearing abutment 101 according to the size of the rock specimen, thereby adjusting the relative position between the rock specimen and the microwave control device 2.

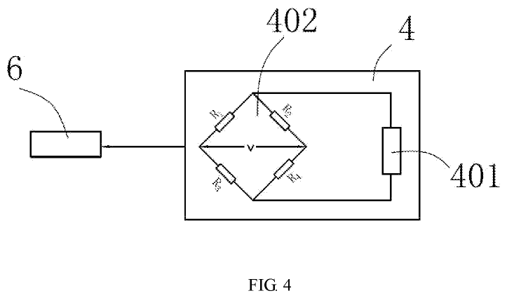

As shown in FIG. 4, the measurement circuit 4 includes an amplifier 401 and a bridge 402. One end of the lead wire 5 is connected to the strain measurement device 3, and the other end of the lead wire 5 is connected to the bridge 402 through the amplifier 401, and the bridge 402 is connected with the resistance strain gauge 6. The electric signal collected by the strain measurement device 3 is processed by the amplifier 401 and the bridge 402, which is favorable for improving the reliability of the test data.

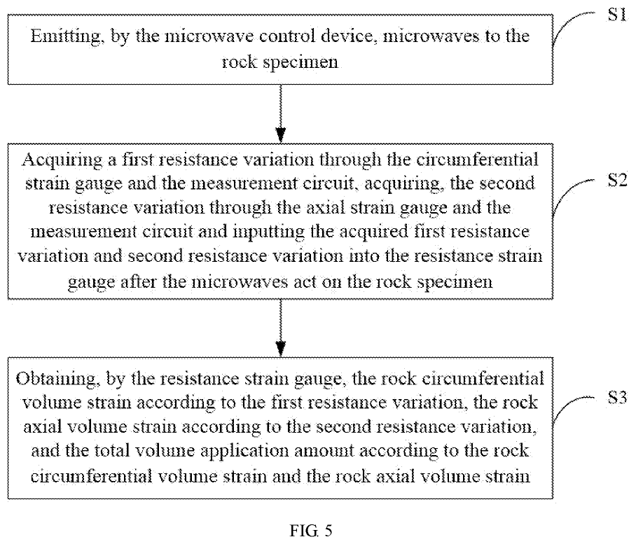

As shown in FIG. 5, a method for measuring the rock volume change under microwave action is based on the above measurement system for rock volume change, and the method includes the following steps:

S1. emitting, by the microwave control device 2, microwaves to the rock specimen.

S2. acquiring a first resistance variation through the circumferential strain gauge 301 and the measurement circuit 4, acquiring, the second resistance variation through the axial strain gauge 302 and the measurement circuit 4 and inputting the acquired first resistance variation and second resistance variation into the resistance strain gauge 6 after the microwaves act on the rock specimen.

S3. obtaining, by the resistance strain gauge 6, the rock circumferential volume strain according to the first resistance variation, the rock axial volume strain according to the second resistance variation, and the total volume application amount according to the rock circumferential volume strain and the rock axial volume strain.

It should be noted that the function of the measurement circuit 4 and the resistance strain gauge 6 is to automatically convert the resistance variation into the volume variation, which is a conventional application of the prior art to the measurement circuit 4 and the resistance strain gauge 6. Taking five circumferential strain gauges 301 and five axial strain gauges 302 as an example, the specific process of analyzing the volume change of rock is described.

Assuming that the original height of the rock specimen is l, the cross-sectional cylindrical radius is r, a cylinder element is taken in the rock specimen, and the side length of each side is dl, dr, then the volume of the element before deformation is dV For dV=πdl·(dr)².

The axial strain measured by each strain gauge after deformation is εᵢ, the circumferential strain εᵢ (=1, 2, 3, 4, 5).

Each segment of deformation can be considered as approximately uniform deformation, and the length change of each side after deformation is: (1+εᵢ) dl, (1+εᵢ′) dr.

The volume of the deformed infinitesimal body is dV=π(1+εᵢ) dl·(1+εᵢ′)²(dr)², then the volumetric strain θ is

$$θ = \frac{π ( 1 + {ɛ}_{i} ) dl \cdot {( 1 + {ɛ}_{i}^{′} )}^{2} dr - π dl \cdot {( dr )}^{2}}{π dl \cdot {( dr )}^{2}} .$$

Expanding the above calculation formula and neglecting the higher order trace, θᵢ=εᵢ+2εᵢ′ is obtained.

Let the original volume of each segment be set to Vᵢ⁰, the volume of each segment after deformation is Vᵢ, the total volume of the rock specimen is

$$V = {\sum }_{i = 1}^{5} ( 1 + {θ}_{i} ) {V}_{i}^{0} ,$$

wherein, (i=1, 2, 3, 4, 5).

In conclusion, the disclosure provides a system and method for measuring the volume change of rock under the action of microwave, which includes a test cavity 1, a microwave control device 2, a strain measurement device 3, a measurement circuit 4 and a resistance strain gauge 6. The test cavity 1 is of a sealed cavity structure, and a rock specimen is placed in the test cavity 1. The microwave control device 2 is arranged inside the test cavity 1. The microwave control device 2 is used for applying microwaves on the rock specimen. The strain measurement device 3 includes a number of circumferential strain gauges 301 and a number of axial strain gauges 302, and the number of strain measurement device 3 are attached to the surface of the rock specimen. The measurement circuit 4 is connected to a number of the strain measurement devices 3 through lead wires 5, and the resistance strain gauges 6 are connected to the measurement circuit 4. In the disclosure, the non-uniform deformation generated after the rock specimen is heated by the microwave is considered, the annular deformation and the axial deformation of each section of the rock specimen are measured in a sectional manner, and the deformation of each section of the stone test piece is approximately uniform deformation, thereby obtaining the whole volume of the deformed rock specimen, eliminating the volume change of liquid or gas caused by temperature change, and more accurately measuring the influence of the microwave action on the rock volume.

It is to be understood that the application of the present invention is not limited to the above examples, and that modifications and variations can be made to those skilled in the art in light of the above description, and all such modifications and variations are intended to fall within the scope of the appended claim.

What is claimed is:

1. A measurement system for rock volume change under microwave action, comprising:
a test cavity of a sealed cavity structure; a rock specimen being placed in the test cavity;
a microwave control device provided inside the test cavity for applying microwaves on the rock specimen;
a plurality of strain measurement devices comprising a plurality of circumferential strain gauges and a plurality of axial strain gauges, and the plurality of strain measuring devices being attached to surfaces of the rock specimen;
a measurement circuit and a resistance strain gauge, wherein the measurement circuit is connected with the plurality of the strain measuring devices through lead wires, and the resistance strain gauge is connected with the measurement circuit.

2. The measurement system for rock volume change under microwave action of claim 1, wherein the strain measuring device comprises sensitive grids, a substrate and a covering layer;
a sheet structure is defined by the sensitive grids, the substrate and the covering layer, the sensitive grids are arranged between the substrate and the covering layer and are connected with the measurement circuit through the lead wires;
the substrate is arranged close to the rock specimen, and the covering layer is arranged far away from the rock specimen when the strain measuring device is attached to a surface of the rock specimen.

3. The measurement system for rock volume change under microwave action of claim 2, wherein the covering layer is a foamed silicon dioxide layer.

4. The measurement system for rock volume change under microwave action of claim 2, wherein the sensitive grids are arranged along the horizontal direction in the circumferential strain sheet; and the sensitive grids are arranged along the vertical direction in the axial strain sheet.

5. The measurement system for rock volume change under microwave action of claim 4, wherein the number of the circumferential strain gauges is 3 to 10, and the sensitive grids between the circumferential strain gauges are parallel to each other, and the number of the axial strain gauges is 3 to 10, and the sensitive grids between the axial strain gauges are parallel to each other.

6. The measurement system for rock volume change under microwave action of claim 1, wherein the microwave control device comprises a microwave source, a wave-guide assembly, and a microwave emission disk; one end of the microwave source is connected with the wave-guide assembly, and the other end of the microwave source is connected with the microwave emission disk;
the test cavity includes a bottom plate, a side plate and a top plate, wherein a rock bearing abutment is arranged on the bottom plate of the test cavity, and a rock specimen is placed on the rock bearing abutment;
the microwave source is arranged on the top plate or the side plate of the test cavity, and the microwave emission disk is configured to emit microwaves towards the rock specimen.

7. The measurement system for rock volume change under microwave action of claim 6, wherein the microwave source comprises a power supply assembly, a transformer assembly and a control circuit.

8. The measurement system for rock volume change under microwave action of claim 6, wherein the bottom plate, the side plate and the top plate are plate-shaped double-layer structures consisting of a metal layer cavity and a heat insulation layer cavity;
the side plate is provided with a pick-and-place through hole, and the pick-and-place through hole is provided with the side door;
the rock bearing abutment is arranged on the bottom plate through a height adjust assembly.

9. The measurement system for rock volume change under microwave action of claim 1, wherein the measurement circuit comprises an amplifier and a bridge;
one end of each of the lead wires is connected with the strain measuring device, and each other end of the lead wires is connected with the bridge through the amplifier, and the bridge is connected with the resistance strain gauge.

10. A method for measuring a rock volume change under an action of microwaves, comprising:
emitting, by the microwave control device, microwaves to the rock specimen;
acquiring a first resistance variation through the circumferential strain gauge and the measurement circuit, acquiring, the second resistance variation through the axial strain gauge and the measurement circuit and inputting the acquired first resistance variation and second resistance variation into the resistance strain gauge after the microwaves act on the rock specimen; and
obtaining, by the resistance strain gauge, the rock circumferential volume strain according to the first resistance variation, the rock axial volume strain according to the second resistance variation, and the total volume application amount according to the rock circumferential volume strain and the rock axial volume strain.
